(19) United States
(12) Patent Application Publication
(10) Pub. No.: US 2026/0226416 A1
(43) Pub. Date: Aug. 6, 2026

(54) METHOD FOR PREPARING CO-DIFFERENTIATED CELL THERAPEUTIC AGENT

(71) Applicant: THE CATHOLIC UNIVERSITY OF KOREA INDUSTRY ACADEMIC COOPERATION FOUNDATION, Seoul (KR)

(72) Inventors: Eun Hu LEE, Seoul (KR); Seung Yeon JEONG, Seoul (KR); Jun Hee CHOI, Seoul (KR); Huijin LIM, Daejeon (KR)

(21) Appl. No.: 19/462,603

(22) Filed: Jan. 28, 2026

(30) Foreign Application Priority Data
Feb. 4, 2025 (KR) 10-2025-0013480

(51) Int. Cl. C12N 5/077

(52) U.S. Cl. CPC C12N 5/0658; C12N 2502/1335

(57) ABSTRACT

The present invention relates to a method for preparing a co-differentiated cell therapeutic agent, and more specifically, since the cell therapeutic agent of the present invention is prepared through the co-differentiation of immature myotubes differentiated in vitro, the processes of proliferation and early terminal differentiation of cells transplanted at a transplanted site after transplantation can be omitted, a solution to the problems of satellite stem cells, which have quantitative disadvantages, or myoblasts, which have low success rates in proliferation and early terminal differentiation of cells transplanted at a transplanted site after the transplantation process, is provided, and skeletal muscle contractility (skeletal muscle physiological activity) can be increased by enhancing the ability to procure calcium used for skeletal muscle contraction at the transplanted site.

### CROSS-REFERENCE TO RELATED APPLICATION

This application claims priority to and the benefit of Korean Patent Application No. 2025-0013480, filed on Feb. 4, 2025, the disclosure of which is incorporated herein by reference in its entirety.

### BACKGROUND

### 1. Field of the Invention

The present invention relates to a method for preparing a co-differentiated cell therapeutic agent.

This work was fully supported (100% contribution) by a grant from the Ministry of Science and ICT (MSIT), Republic of Korea, through the National Research Foundation of Korea (NRF), under the Basic Science Research Program for Mid-career Researchers (NRF-2022R1A2C1005362/RS-2022-NR069238). The project was conducted by The Catholic University of Korea Industry-Academic Cooperation Foundation, with Eun Hui Lee as the principal investigator, during the research period from Mar. 1, 2022 to Feb. 28, 2027, and entitled “Presentation of the pathogenesis and therapeutic strategies of Muscular Dystrophy”.

### 2. Discussion of Related Art

To resolve skeletal muscle atrophy/wasting caused by genetic or acquired skeletal muscle diseases, skeletal muscle atrophy/wasting that occurs during the long-term treatment of other diseases (particularly cancer treatment, and the like), loss of skeletal muscle caused by fire or physical accidents, skeletal muscle atrophy/wasting due to abnormalities or loss of a nerve innervating skeletal muscle, skeletal muscle atrophy/wasting due to insufficient nutrient intake such as malnutrition or fasting, skeletal muscle atrophy/wasting caused by infrequent use of skeletal muscle such as maintaining a sedentary posture for long periods of time due to occupation or lifestyle, skeletal muscle atrophy/wasting caused by weightlessness in astronauts and the like, sarcopenia of skeletal muscle that occurs during the natural aging process, or the like, skeletal muscle cell transplantation is being attempted at the corresponding sites.

In this process, although ‘satellite cells’ isolated from tissues or ‘myoblasts’ obtained through cell culture are used as transferred cells, there is a disadvantage in that satellite cells are difficult to obtain quantitatively due to their small quantity and myoblasts have low success rates in proliferation and early terminal differentiation of cells transplanted at a transplanted site after transplantation.

### SUMMARY OF THE INVENTION

The present invention is directed to a cell therapeutic agent having a high success rate of transplantation in preventing or treating muscle diseases associated with skeletal muscle atrophy/wasting, a method for preparing the same, and a use thereof.

The present invention is also directed to D2-to-D3 mature co-differentiation myotubes that have increased expression of MyoD, MyHC II, RyR1, SERCA1, and STIM1 proteins, increased cell thickness, and increased intracellular or extracellular calcium procurement for skeletal muscle contraction compared to control myotubes obtained by differentiating myoblasts in vitro for 5 days. According to an aspect of the present invention, there is provided a method for preparing the D2-to-D3 mature co-differentiation myotubes, the method includes co-differentiating D2 immature myotubes and D3 immature myotubes, wherein the D2 immature myotubes obtained on day 2 after initiation of terminal differentiation of myoblasts are used as transferred cells, and the D3 immature myotubes obtained on day 3 are used as host cells.

According to another aspect of the present invention, there is provided a pharmaceutical composition for preventing or treating a muscle disease associated with skeletal muscle atrophy/wasting, containing the D2-to-D3 mature co-differentiation myotubes as an active ingredient.

According to another aspect of the present invention, there is provided a method for treating a muscle disease associated with skeletal muscle atrophy/wasting, the method including administering, to a subject in need thereof, a therapeutically effective amount of the pharmaceutical composition according to the present invention.

Since the cell therapeutic agent of the present invention is prepared through the co-differentiation of immature myotubes differentiated in vitro, the processes of proliferation and early terminal differentiation of cells transplanted at a transplanted site after transplantation can be omitted, a solution to the problems of satellite stem cells, which have quantitative disadvantages, or myoblasts, which have low success rates in proliferation and early terminal differentiation of cells transplanted at a transplant site after transplantation, is provided, and skeletal muscle contractility (skeletal muscle physiological activity) can be increased by enhancing the ability to procure calcium used for skeletal muscle contraction at the transplanted site.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a schematic view of the co-differentiation strategy during terminal differentiation of myoblasts into myotubes;

FIG. 2A-B shows the results of comparing the degree of intracellular calcium transport for skeletal muscle contraction in co-differentiated myotubes (A: control, Mb-to-D2, D2-to-D2, D3-to-D2; B: control, Mb-to-D3, D2-to-D3, D3-to-D3);

FIG. 3A-D shows the results of comparing the cell widths of D2-to-D3 co-differentiated myotubes and the expression levels of key proteins involved in skeletal muscle contraction and relaxation on day 5 of differentiation: (A) Results of measuring the width of D2-to-D3 co-differentiated myotubes on day 5 of differentiation. (B) Results of comparing the expression levels of MyoD and myogenin (terminal differentiation-associated protein), and MyHC II (contraction-mediating protein) in D2-to-D3 co-differentiated myotubes on day 5 of differentiation. (C) Results of comparing the expression levels of RyR1, DHPR, SERCA1a, and CASQ1, which are key proteins mediating skeletal muscle contraction and relaxation. (D) Results of performing immunoblot analysis to compare the expression levels of triad formation-mediating proteins; and

FIG. 4A-D shows the results of comparing the store-operated calcium entry (SOCE) that mediates external calcium procurement during skeletal muscle contraction in D2-to-D3 co-differentiated myotubes, the cytosolic calcium concentrations, the amounts of calcium stored in the sarcoplasmic reticulum, and the expression levels of SOCE-mediating proteins on day 5 of differentiation. (A) Results of measuring, after depleting the amount of calcium stored in the sarcoplasmic reticulum (SR) by thapsigargin (TG) treatment, the resulting store-operated calcium entry (SOCE). (B) Results of comparing the amounts of calcium in the cytosol and (C) results of comparing the amounts of calcium stored in the sarcoplasmic reticulum (or the amounts of calcium that can be released from the sarcoplasmic reticulum into the cytosol, that is, the amounts of calcium that can be used for skeletal muscle contraction) by thapsigargin (TG) treatment. (D) Results of comparing the expression levels of SOCE-mediating proteins using immunoblotting (TRPC: transient receptor potential cation channel, STIM).

### DETAILED DESCRIPTION OF EXEMPLARY EMBODIMENTS

Hereinafter, the configuration of the present invention will be described in detail.

The present invention relates to D2-to-D3 mature co-differentiation myotubes that have increased expression of MyoD, MyHC II, RyR1, SERCA1, and STIM1 proteins, increased cell thickness, and increased intracellular or extracellular calcium procurement for skeletal muscle contraction compared to control myotubes obtained by differentiating myoblasts in vitro for 5 days.

Further, the present invention provides a method for preparing the D2-to-D3 mature co-differentiation myotubes, the method includes co-differentiating D2 immature myotubes and D3 immature myotubes, wherein the D2 immature myotubes obtained on day 2 after initiation of terminal differentiation of myoblasts are used as transferred cells, and the D3 immature myotubes obtained on day 3 are used as host cells.

As used herein, the term “terminal differentiation of myoblasts” refers to the process by which mononuclear myoblasts fuse to form multinucleated myotubes. Myoblasts, which correspond to the precursor cells of skeletal muscle, may be distinguished using a Pax7⁺ marker in the case of self-renewal, and may be distinguished by Pax7⁺/MyoD⁺ in the case of proliferation. In addition, cells that form myotubes at the differentiation stage may be distinguished using a Pax7⁻ MyoD⁺ MyoG⁺ marker. The cells that form myotubes at the early stage of differentiation show increased expression of myogenic transcription factors such as myosin D (MyoD), and at the intermediate stage, myogenin is increased. At the later stage when differentiation is almost complete, the expression of myosin heavy chain, a protein that directly participates in skeletal muscle contraction, is increased.

In order to overcome the disadvantages of satellite stem cells and myoblasts according to cell transplantation when treating muscle diseases associated with skeletal muscle atrophy/wasting, the inventors used immature myotubes that had undergone partial terminal differentiation as transferred cells and also used immature myoblasts as host cells and allowed them to co-differentiate, and as a result, the inventors obtained D2-to-D3 mature co-differentiation myotubes that have increased expression of MyoD, MyHC II, RyR1, SERCA1, and STIM1 proteins, increased cell thickness, and increased intracellular or extracellular calcium procurement for skeletal muscle contraction compared to control myotubes obtained by differentiating myoblasts in vitro for 5 days.

Specifically, the D2-to-D3 mature co-differentiation myotubes of the present invention may be obtained using D2 immature myotubes obtained on day 2 after the initiation of terminal differentiation of myoblasts as transferred cells and D3 immature myotubes on day 3 as host cells to transfer the D2 immature myotubes to the D3 immature myotubes, thereby allowing them to co-differentiate.

As used herein, the term “transferred cells” refers to any cells that are transferred into a culture environment of other cells for co-culture.

As used herein, the term “host cells” refers to any other cells that provide a culture environment for the transferred cells in the co-culture.

As the myoblasts, it is possible to use mouse myoblasts, rat myoblasts, frog myoblasts, rabbit myoblasts, guinea pig myoblasts, porcine myoblasts, monkey myoblasts, human myoblasts, or the like, but the myoblasts are not limited thereto.

The co-differentiation may be performed by culturing D2 immature myotubes on day 2 and D3 immature myotubes on day 3 together in vitro for two additional days, but is not limited thereto.

Furthermore, differentiation and co-differentiation of myoblasts may be performed in a differentiation medium.

As used herein, the term “medium” refers to a mixture for the growth and proliferation of cells in vitro, containing elements essential for the growth, proliferation and the like of cells, such as sugars, amino acids, various nutrients, serum, growth factors, and minerals. In particular, the medium of the present invention is a medium for differentiation and co-differentiation of myoblasts, including cell growth and proliferation. The medium may be prepared by adding supplementary components for differentiation or co-differentiation to various basal media. The “basal medium” is a mixture containing essential sugars, amino acids, water, and the like required for cell survival, and may be artificially prepared and used, or a commercially prepared medium may be used. Examples of the commercially prepared medium include, for example, Dulbecco's Modified Eagle's Medium (DMEM), Minimal Essential Medium (MEM), Basal Medium Eagle (BME), RPMI 1640, F-10, F-12, α-Minimal Essential Medium (α-MEM), Glasgow's Minimal Essential Medium (G-MEM), Iscove's Modified Dulbecco's Medium, and the like, but are not limited thereto.

Specifically, in the differentiation medium of the present invention, 5% heat-inactivated horse serum and low-glucose DMEM are used instead of 20% FBS and F-10 Nutrient Mixture, and bFGF is not used.

The differentiation or co-differentiation may be performed in a 10% CO₂ incubator.

In the transplantation of myotubes into sites of skeletal muscle atrophy/wasting caused by genetic or acquired skeletal muscle diseases, skeletal muscle atrophy/wasting that occurs during the long-term treatment of other diseases, or sarcopenia of skeletal muscle that occurs in the natural aging process, the D2-to-D3 mature co-differentiation myotubes are prepared through the co-differentiation of immature myotubes differentiated in vitro, so that the processes of proliferation and early terminal differentiation of cells transplanted at a transplanted site after transplantation may be omitted, a solution to the problems of satellite stem cells, which have quantitative disadvantages, or myoblasts, which have low success rates in proliferation and early terminal differentiation of cells transplanted at a transplanted site after transplantation, is provided, and skeletal muscle contractility (skeletal muscle physiological activity) may be increased by enhancing the ability to procure calcium for skeletal muscle contraction at the transplanted site.

Therefore, the D2-to-D3 mature co-differentiation myotubes of the present invention may be used as a cell therapeutic agent for preventing or treating muscle diseases associated with skeletal muscle atrophy/wasting.

Accordingly, the present invention provides a pharmaceutical composition for preventing or treating a muscle disease associated with skeletal muscle atrophy/wasting, containing the D2-to-D3 mature co-differentiation myotubes as an active ingredient.

The muscle diseases associated with skeletal muscle atrophy/wasting are muscle diseases derived from atrophy or damage to muscle tissue due to genetic, pathological or physical causes.

Primary skeletal muscle atrophy/wasting may result directly from a variety of hereditary muscle disorders, including congenital and hereditary myopathies. Hereditary myopathies are associated with progressive atrophy, inflammation, muscle fiber metabolic disorders, muscle spasms, or stiffness. Hereditary myopathies may be further subdivided into muscular dystrophies, congenital myopathies, mitochondrial myopathies, and metabolic myopathies. The more common hereditary myopathies are mitochondrial and metabolic myopathies. Congenital myopathies include nemaline myopathy, and muscular dystrophies include Duchenne muscular dystrophy, Becker muscular dystrophy, and myotonic dystrophy (types 1 and 2).

In contrast, acquired causes such as systemic diseases or physical conditions may cause secondary skeletal muscle atrophy/wasting. Pathological conditions that induce skeletal muscle atrophy/wasting include sarcopenia associated with aging, cancer-induced cachexia, chronic obstructive pulmonary disease, diabetes and obesity, chronic kidney disease, heart failure, neurodegenerative diseases, sepsis, burns, and trauma. Physiological responses such as fasting or malnutrition may also cause skeletal muscle atrophy/wasting. Skeletal muscle atrophy/wasting may occur in patients who are immobilized, such as those with leg fractures, disuse, immobilization and bed rest, and may also occur in ordinary people who maintain weightlessness, live a simply sedentary lifestyle, or have occupations that require long periods of sitting. Ultimately, skeletal muscle atrophy/wasting signaling is initiated by a lack of muscle contraction and stimulation due to various causes, thereby causing protein loss and cell death. As such diseases progress, muscle atrophy/wasting occurs when the rate of protein breakdown exceeds the rate of protein synthesis.

Sarcopenia refers to the gradual age-related reduction in skeletal muscle mass and strength in adults. Skeletal muscle atrophy/wasting and fatigue due to aging is a long-term process, the causes of which are multifactorial and complex, involving both external and internal factors. Since aged skeletal muscle exhibits slow metabolism, reduced biosynthesis, and a small mitochondrial size, muscle mass and efficiency parameters are rapidly lost. The aging process reduces average muscle capacity, leading to instability, increased risk of falls, and bone fractures, with a corresponding increased need for health and social services.

Specifically, examples of the muscle diseases associated with skeletal muscle atrophy/wasting of the present invention include muscular atrophy, myasthenia, muscular dystrophy, myotonia, muscular hypotonia, muscle weakness, muscle degenerative atrophy, amyotrophic lateral sclerosis or myasthenia gravis, central core disease due to genetic mutations, malignant hyperthermia, Duchenne muscular dystrophy, Becker muscular dystrophy, or the like, but are not limited thereto.

Further, examples of muscular atrophy include sarcopenia, disuse atrophy, muscular atrophy caused by absence of mechanical stimulation, denervation atrophy, drug-induced atrophy, malnutritional atrophy, muscular dystrophy, or the like, but are not limited thereto.

As used herein, the term “prevention” refers to any action that suppresses or delays the onset of skeletal muscle atrophy/wasting by administering the composition according to the present invention, and “treatment” refers to any action that ameliorates or beneficially changes the symptoms of individuals suspected of or suffering from skeletal muscle atrophy/wasting by administering the pharmaceutical composition.

The pharmaceutical composition of the present invention may contain a therapeutically effective amount of D2-to-D3 mature co-differentiation myotubes for the treatment of muscle diseases associated with skeletal muscle atrophy/wasting. The “therapeutically effective amount” refers to an amount of active ingredient or pharmaceutical composition that is considered by a researcher, veterinarian, physician or other clinicians to induce a biological or medical response in a tissue system, animal or human, and this includes an amount that induces alleviation of the symptoms of a disease or disorder being treated. It is obvious to those skilled in the art that the content (number) of D2-to-D3 mature co-differentiation myotubes contained in the pharmaceutical composition of the present invention will vary depending on the desired effect. Therefore, an optimal content of the cellular therapeutic agent may be easily determined by those skilled in the art, and may be adjusted by various factors including the type of disease, the severity of disease, the content of other ingredients contained in the composition, the type of dosage form, and the age, body weight, general health status, gender and diet of a patient, the administration time, the administration route and the excretion rate of a composition, treatment period, and simultaneously used drugs, but, for example, 1×10⁴ cells/kg to 1×10⁸ cell/kg of D2-to-D3 mature co-differentiation myotubes may be contained, but is not limited thereto.

In the present invention, the composition may be characterized in that it is in the form of a capsule, a tablet, a granule, an injection, an ointment, a powder, or a beverage, and the composition may be characterized in that it is preferably intended for humans.

The pharmaceutical composition of the present invention may be used by being formulated into the form of an oral dosage form such as powder, granules, a capsule, a tablet, and an aqueous suspension, an external preparation, a suppository, and a sterile injectable solution, but is not limited thereto. The pharmaceutical composition of the present invention may include a pharmaceutically acceptable carrier. As the pharmaceutically acceptable carrier, a binder, a lubricant, a disintegrant, an excipient, a solubilizer, a dispersant, a stabilizer, a suspending agent, a colorant, a flavoring agent, and the like may be used when orally administered, in the case of injection, a buffer, a preservative, an analgesic, a solubilizer, an isotonic agent, a stabilizer, and the like may be mixed and used, and in the case of topical administration, a base, an excipient, lubricant, a preservative, and the like may be used. The formulation of the pharmaceutical composition of the present invention may be prepared in various ways by mixing the pharmaceutical composition of the present invention with a pharmaceutically acceptable carrier as described above. For example, the formulation may be prepared in the form of a tablet, a troche, a capsule, an elixir, a suspension, a syrup, a wafer, and the like when orally administered, and in the case of injection, the injection may be formulated into unit dosage ampoules or in multiple dosage forms. The pharmaceutical composition of the present invention may be formulated into other solutions, suspensions, tablets, capsules, sustained-release preparations, and the like.

Meanwhile, as an example of suitable carriers, excipients and diluents for formulation, it is possible to use lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, or the like. Further, the pharmaceutical composition of the present invention may further include a filler, an anticoagulant, a lubricant, a wetting agent, a flavoring agent, an emulsifier, an antiseptic, and the like.

The route of administration of the pharmaceutical composition according to the present invention includes, but is not limited to, oral, intravenous, intramuscular, intraarterial, intramedullary, intrathecal, intracardiac, transdermal, subcutaneous, intraperitoneal, intranasal, enteral, topical, sublingual or rectal administration. Oral or parenteral administration is preferred.

In the present invention, the “parenteral” includes subcutaneous, intradermal, intravenous, intramuscular, intraarticular, intrasynovial, intrasternal, intrathecal, intralesional and intracranial injection or infusion techniques.

The pharmaceutical composition of the present invention may vary depending on various factors including the activity of the specific compound used, age, body weight, general health, sex, diet, time of administration, route of administration, rate of excretion, drug combination and the severity of a particular disease to be prevented or treated, and the dosage of the pharmaceutical composition varies depending on the condition and body weight of the patient, the degree of disease, the form of drug, the route of administration and duration, but may be appropriately selected by a person skilled in the art, and may be 0.0001 to 50 mg/kg or 0.001 to 50 mg/kg daily. The administration may be carried out once daily, or may be divided into several times. The dosage is not intended to limit the scope of the present invention in any way. The pharmaceutical composition according to the present invention may be formulated into pills, dragees, capsules, solutions, gels, syrups, slurries, and suspensions.

The pharmaceutical composition of the present invention can be used alone or in combination with conventional therapeutic agents for muscle diseases associated with skeletal muscle atrophy/wasting, and the like, or may be used in combination with surgery, radiation therapy, hormone therapy, chemotherapy, and methods using biological response modifiers, or exercise therapy or rehabilitation therapy, and the like.

The present invention also provides a method for treating a muscle disease associated with skeletal muscle atrophy/wasting, the method including administering, to a subject in need thereof, a therapeutically effective amount of the pharmaceutical composition according to the present invention.

As used herein, the term “subject in need thereof” refers to a patient suffering from or suspected of having a muscle disease associated with skeletal muscle atrophy/wasting.

As used herein, the term “subject” refers to a human or animal such as a monkey, dog, goat, pig, mouse, rat, guinea pig, rabbit, or primate, having a disease whose symptoms are ameliorated by administering the pharmaceutical composition of the present invention. The pharmaceutical composition of the present invention may be applied not only to humans (therapeutic, suppressive or prophylactic) but also to other commercially useful animals.

In the treatment method of the present invention, when the pharmaceutical composition of the present invention is administered once or several times a day, the cell therapeutic agent (D2-to-D3 mature co-differentiation myotubes) contained in the composition is preferably contained in an amount of 1×10⁴ cells/kg to 1×10⁸ cells/kg.

In the treatment method of the present invention, the pharmaceutical composition of the present invention may be administered in a typical manner via rectal, intravenous, intraarterial, intraperitoneal, intramuscular, intrasternal, transdermal, topical, intraocular or intradermal routes.

Hereinafter, one or more specific exemplary embodiments will be described in more detail through examples. However, these examples are provided only for exemplarily describing the one or more specific exemplary embodiments, and the scope of the present invention is not limited to these examples.

### <Example 1> Preparation of Co-Differentiated Myotubes

To overcome the shortcomings of satellite stem cells and myoblasts, the present inventors conducted experiments to confirm (1) whether the success rate of transplantation could be increased, (2) whether further differentiation would occur well upon transplantation to produce differentiated mature myotubes, and (3) whether the differentiated mature myotubes would exhibit high physiological activity for skeletal muscle contraction, when immature myotubes that have undergone partial terminal differentiation are used as transferred cells, and immature myotubes are also used as host cells (that is, in a state in which immature myotubes are contained or maintained because skeletal muscle atrophy/wasting or sarcopenia cannot be resolved by the tissue itself) for co-differentiation.

(Method for Isolating Skeletal Muscle Satellite Cells and Differentiating them into Myotubes)

Skeletal muscle satellite cells were isolated from the skeletal muscle of fetal mice and subjected to primary culture to obtain myoblasts.

The cells were treated with a cell culture solution (F10 Nutrient Mixture composition: 20% FBS, 100 units/ml penicillin, 100 μg/ml streptomycin, 2 mM L-glutamine, 20 nM basic fibroblast growth factor) and cultured at 37° C. in a 5% CO₂ incubator. Depending on the use, 10-cm, 96-well, or 6-well culture dishes were used, and all the dishes were coated with Matrigel and used. When the cells proliferated to reach approximately 65% confluency in the culture dish, the cells were induced to differentiate into myotubes (Differentiation medium composition: 5% heat-inactivated horse serum and low-glucose DMEM were used in place of 20% FBS and F-10 Nutrient Mixture, bFGF was omitted, and the cells were cultured in a 10% CO₂ incubator).

### (Method for Co-Differentiation of Skeletal Muscle Satellite Cells)

Myoblasts obtained by primary culture were differentiated in the differentiation medium composition for 2 or 3 days to prepare immature myotubes (D2 or D3 immature myotubes). Non-co-differentiated cells used as a control were differentiated in the differentiation medium composition for 5 days as usual to obtain mature myotubes.

Three cells to be used as transferred cells (myoblasts, D2 immature myotubes, and D3 immature myotubes) were harvested on days 0, 2, and 3 of differentiation, respectively, and transferred to host cell culture dishes containing D2 immature myotubes or D3 immature myotubes to be used as host cells. Thereafter, the transferred cells and host cells were co-differentiated until day 5 of differentiation based on the host cells to obtain mature co-differentiated myotubes.

The co-differentiated myotubes obtained in this way were named Mb-to-D2, D2-to-D2, D3-to-D2, Mb-to-D3, D2-to-D3, and D3-to-D3 co-differentiated myotubes, respectively.

### (Method for Measuring Co-Differentiated Myotube Width)

Using an inverted light microscope (Nikon Eclipse TS100, Nikon Instruments, Inc.) equipped with a camera (monochrome camera, ProgRes MF, JENOPTIK Optical Systems, Inc.), magnified images of the co-differentiated myotubes present within randomly selected sections of a specific size (1,000 μm wide and 600 μm long, PregRes Capture Pro v2.8.8, JENOPTIK Optical Systems, Inc.) were obtained, and the length of the thickest part of the co-differentiated myotubes was measured using the ImageJ program.

### (Method of Obtaining Lysates of Co-Differentiated Myotubes)

Co-differentiated myotubes were harvested and solubilized in a lysis solution (1% Triton X-100, 10 mM Tris-HCl, 1 mM Na₃VO₄, 10% glycerol, 150 mM NaCl, 5 mM EDTA, protease inhibitor cocktail, pH 7.4) at 4° C. for 24 hours to obtain lysates of co-differentiated myotubes.

### (Immunoblotting)

The lysates of co-differentiated myotubes were separated on a 10% or 12% SDS-PAGE gel, proteins separated on the gel were transferred to a polyvinylidene fluoride (PVDF) membrane (100 V, 2 hours), and the membrane was treated with 5% non-fat milk for 1 hour, and then treated with the corresponding primary antibody (1:1000), subsequently treated with the corresponding horseradish peroxidase-conjugated secondary antibody for 45 minutes, and then visualized and analyzed by colorimetric reaction (SuperSignal ultrachemiluminescent substrate). Information on the antibodies used is shown in Table 1. The antibodies were used at a dilution of 1:1,000. However, the secondary antibody was used at a dilution of 1:50,000.

TABLE 1
Antibody information

| | Antibody | Ordering |
|---|---|---|
| Antibody name | manufacturer name | information |
| RyR1 antibody | Thermo Fisher Scientific | MA3-925 |
| DHPR antibody | (Waltham, MA, USA) | MA3-920 |
| SERCA1a antibody | | MA3-912 |
| CASQ1 antibody | | MA3-913 |
| JP1 antibody | | 40-5100 |
| TRPC1 antibody | Alomone Labs | ACC-010 |
| TRPC3 antibody | (Jerusalem, Israel) | ACC-016 |
| TRPC4 antibody | | ACC-018 |
| TRPC6 antibody | | ACC-017 |
| Orai1 antibody | Santa Cruz Biotechnology | sc-377281 |
| α-actin antibody | (Dallas, TX, USA) | sc-58671 |
| MyoD antibody | | sc-377460 |
| myogenin antibody | | sc-398002 |
| JP2 antibody | | sc-134875 |
| MyHC II antibody | Abcam | ab37484 |
| | (Cambridge, MA, USA) | |
| STIM1 antibody | Cell Signaling Technology | 4916 |
| | (Danvers, MA, USA) | |
| STIM2 antibody | Proteintech | 21192-1-AP |
| | (Rosemont, IL, USA) | |
| Goat secondary antibody | Jackson Immuno | 205-035-108 |
| Mouse secondary antibody | Research Laboratories | 715-035-151 |
| Rabbit secondary antibody | (West Grove, PA, USA) | 711035-152 |

### (Measurement of Calcium Responses of Co-Differentiated Myotubes to KCl, Cytosolic Calcium Concentration, and the Amount of Calcium Stored in the Sarcoplasmic Reticulum Using Single-Cell Calcium Imaging Technique)

Fura-2 (5 μM, for measuring cytosolic calcium levels), a calcium fluorescent dye (Ca²⁺ dye) that, when binding to calcium, emits fluorescence with a wavelength different from that before calcium binding, or fluo-4 (5 μM, for other single-cell calcium imaging experiments) was injected into co-differentiated myotubes while being maintained at 37° C. for 45 minutes (incubation). In this case, the co-differentiated myotubes were treated with an imaging solution (125 mM NaCl, 5 mM KCl, 2 mM KH₂PO₄, 2 mM CaCl₂, 25 mM HEPES, 6 mM glucose, 1.2 mM MgSO₄, 0.05% BSA (fraction V), pH 7.4). A fluorescence microscope (Nikon x40 oil-immersion objective, NA 1.30, ECLIPSE Ti, Nikon) was used to measure intracellular calcium movement. The fluorescence changes of the calcium fluorescent dye were transmitted to a computer using a 75-watt Xenon lamp (FSM150Xe, Bentham Instruments, Ltd.) and a 12-bit CCD camera (DVC-340M-OO-CL, Digital Video Camera Company) connected to a fluorescence microscope, and analyzed using an associated program (InCyt Iml image acquisition and analysis software, v5.29, Intracellular Imaging Inc.). Calcium movement from the sarcoplasmic reticulum (SR) to the cytosol by KCl treatment, the cytosolic calcium concentration, and the amount of calcium stored in the sarcoplasmic reticulum (in other words, the amount of calcium that can move from the sarcoplasmic reticulum to the cytosol) by thapsigargin (TG) treatment were measured, and the values corresponding to the peak height of the graph were statistically processed (showing the same tendency as the statistical processing of the values corresponding to the area of the graph). The absolute concentration of calcium in the cytosol was measured using a calcium calibration reagent set (Calcium Calibration Buffer Kit #1, Thermo Fisher Scientific).

### (Measurement of Store-Operated Calcium Entry (SOCE) by Calcium Imaging Technique in Single Myotubes)

Fluo-4 (5 μM), a calcium fluorescent dye (Ca²⁺ dye) that, when binding to calcium, emits fluorescence with a wavelength different from that before calcium binding, was injected into co-differentiated myotubes while being maintained at 37° C. for 45 minutes (incubation). In this case, the myotubes were treated with an imaging solution (125 mM NaCl, 5 mM KCl, 2 mM KH₂PO₄, 2 mM CaCl₂, 25 mM HEPES, 6 mM glucose, 1.2 mM MgSO₄, 0.05% BSA (fraction V), pH 7.4). A fluorescence microscope (Nikon x40 oil-immersion objective, NA 1.30, ECLIPSE Ti, Nikon) was used to measure calcium transfer in the co-differentiated myotubes. The fluorescence changes of the calcium fluorescent dye were transmitted to a computer using a 75-watt Xenon lamp (FSM150Xe, Bentham Instruments, Ltd.) and a 12-bit CCD camera (DVC-340M-OO-CL, Digital Video Camera Company) connected to a fluorescence microscope, and analyzed using an associated program (InCyt Iml image acquisition and analysis software, v5.29, Intracellular Imaging Inc.). For an experiment of measuring the amount of calcium that flows from the outside of the cell to the inside of the cell (store-operated Ca²⁺ entry (SOCE)) when calcium in the calcium store (that is, the sarcoplasmic reticulum (SR)) is depleted, myotubes were treated with a calcium (Ca²⁺)-free imaging solution for 5 minutes, and then treated with thapsigargin to induce the depletion of calcium in the sarcoplasmic reticulum, and the outside of the cell was treated with 2 mM calcium to measure the amount of calcium that flows into the outside of the cell. TG was dissolved in Me₂SO (<0.05%), cells were manually treated with the resulting solution, and it was confirmed that Me₂SO (<0.05%) itself did not alter cellular calcium transport. The results of SOE and TG response measurements were statistically processed using the area of a calcium movement graph.

### (Data Analysis: Statistical Analysis)

All data obtained through multiple experiments was combined and expressed as ±SE. Except for the comparison of cytosolic calcium concentrations, values obtained from the control were set to 1, and relative changes with respect to the values were expressed as normalized ratios. The number of experimental replicates and the number of cells used for data analysis are shown in the drawings or tables. Significant differences were determined using an unpaired t-test or one-way ANOVA-Tukey's post hoc test (GraphPad InStat, v2.04), and significant differences are indicated when P<0.05 (* or #). Graphs were prepared using the Origin 2019b program.

### <Experimental Example 1> Preparation of Myotubes Under Different Co-Differentiation Conditions

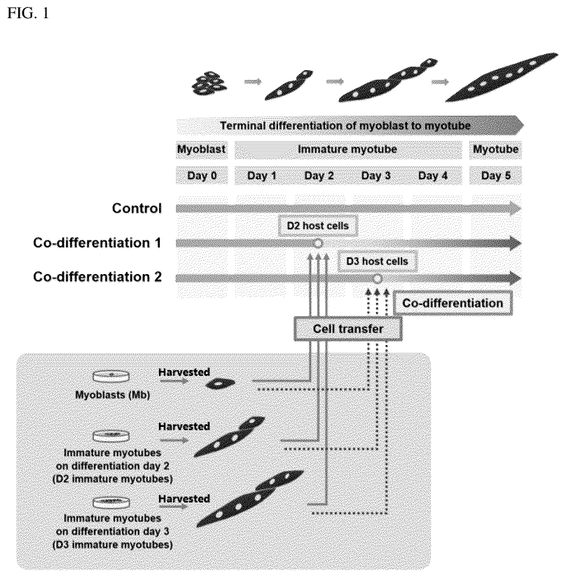

FIG. 1 shows a schematic view of the co-differentiation strategy during terminal differentiation of myoblasts into myotubes.

Days 0 to 5 represent the period from day 0 to day 5 of the initiation of terminal differentiation, myotubes refer to fully differentiated multinucleated myotubes on day 5 of differentiation, D2 or D3 host cells refer to D2 or D3 immature myotubes, co-differentiation condition 1 or 2 refers to the case where D2 or D3 immature myotubes were used as host cells, and the number of cells used under each co-differentiation condition and the cell selection method are shown in Tables 2 and 3.

Table 2 shows the number of myoblasts initially used to obtain myoblasts and immature myotubes under six different conditions (Mb-to-D2, D2-to-D2, D3-to-D2, Mb-to-D3, D2-to-D3, D3-to-D3). All data obtained through multiple experiments (see the following Table 2) was combined and expressed as ±SE. *Significant differences are indicated when P<0.05, compared to Mb-to-D2 (co-differentiation condition 1) or compared to Mb-to-D2 (co-differentiation condition 2). There were no meaningful significant differences.

Table 3 shows the number of spots randomly selected from cell culture dishes and observed to count the number of myoblasts in Table 2, and each spot (1,000 μm wide and 600 μm long) was randomly selected from a different cell culture well.

TABLE 2
Number of myoblasts on day 0 of differentiation
under different co-differentiation conditions

| | | Number of myoblasts on | |
|---|---|---|---|
| Co-differentiation | | day 0 of differentiation | |
| conditions | | Transfer cells | D2 or D3 host cells |
| Co-differentiation | Control | None | 137.53 ± 19.96 |
| condition 1 | Mb-to-D2 | 111.80 ± 22.93 | 112.30 ± 19.94 |
| (D2 host cells) | D2-to-D2 | 99.28 ± 17.90 | 102.73 ± 18.41 |
| | D3-to-D2 | 114.26 ± 20.11 | 108.26 ± 20.90 |
| Co-differentiation | Control | None | 144.49 ± 17.12 |
| condition 2 | Mb-to-D3 | 117.59 ± 19.33 | 101.90 ± 18.75 |
| (D3 host cells) | D2-to-D3 | 92.64 ± 15.96 | 93.87 ± 15.56 |
| | D3-to-D3 | 103.08 ± 18.21 | 104.33 ± 17.76 |

TABLE 3
Number of spots randomly selected from cell culture dishes
and observed to count the number of myoblasts in Table 2

| | |
|---|---|
| Co-differentiation | Number of |
| conditions | targeted spots |

| | | |
|---|---|---|
| Co-differentiation | Control | 120 spots from 10 wells |
| condition 1 | Mb-to-D2 | 192 spots from 16 wells |
| | D2-to-D2 | 240 spots from 20 wells |
| | D3-to-D2 | 192 spots from 16 wells |
| Co-differentiation | Control | 120 spots from 10 wells |
| condition 2 | Mb-to-D3 | 240 spots from 20 wells |
| | D2-to-D3 | 288 spots from 24 wells |
| | D3-to-D3 | 240 spots from 20 wells |

As described above, Mb-to-D2, D2-to-D2, D3-to-D2, Mb-to-D3, D2-to-D3, and D3-to-D3 co-differentiated myotubes were obtained under six different co-differentiation conditions, respectively.

### <Experimental Example 2> Comparison of the Degree of Intracellular Calcium Transfer for Skeletal Muscle Contraction in Co-Differentiated Myotubes

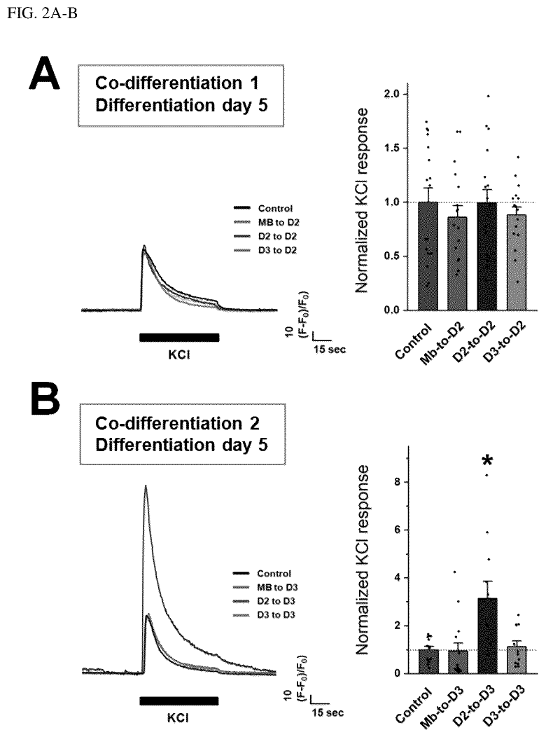

Co-differentiated myotubes were treated with a cell membrane depolarizing agent KCl (a substance that induces excitation-contraction coupling for skeletal muscle contraction) to measure the relative amount of calcium released into the cytosol from the sarcoplasmic reticulum (SR) for skeletal muscle contraction, and the results are shown in FIG. 2. The statistical results regarding this are shown as bar graphs on the right side of FIG. 2. *Significant differences compared to the control are indicated (P<0.05). The number of experiments used for analysis and statistics and the resulting values are shown in Table 4. In Table 4, the data obtained from the number of co-differentiated myotubes shown in parentheses was combined and expressed as ±SE. Values obtained from the control were set to 1, and relative changes with respect to the values were expressed as normalized ratios. *Significant differences compared to the control are indicated when P<0.05.

As illustrated in FIG. 2, it was confirmed that, among the different co-differentiation conditions, intracellular calcium procurement for skeletal muscle contraction was significantly increased in D2-to-D3 co-differentiated myotubes in response to KCl compared to control cells. This suggests that when D2 immature myotubes are used as transferred cells and D3 immature myotubes are used as host cells, the contractile activity of skeletal muscle can be improved through cell transplantation.

TABLE 4
Intracellular calcium movement in response to KCl in co-
differentiated myotubes on day 5 of differentiation.

| Co- | | | | |
|---|---|---|---|---|
| differentiation | | | | |
| condition 1 | Control | Mb-to-D2 | D2-to-D2 | D3-to-D2 |
| Day 5 of | 1.00 ± 0.13 | 0.86 ± 0.11 | 0.99 ± 0.12 | 0.88 ± 0.07 |
| differentiation | (121 myotubes | (122 myotubes | (147 myotubes | (136 myotubes |
| | from 17 wells) | from 17 wells) | from 17 wells) | from 16 wells) |
| Co- | | | | |
| differentiation | | | | |
| condition 2 | Control | Mb-to-D3 | D2-to-D3 | D3-to-D3 |
| Day 5 of | 1.00 ± 0.14 | 0.94 ± 0.34 | 3.15 ± 0.72 * | 1.13 ± 0.23 |
| differentiation | (109 myotubes | (74 myotubes | (84 myotubes | (75 myotubes |
| | from 14 wells) | from 14 wells) | from 12 wells) | from 12 wells) |

### <Experimental Example 3> Comparison of Cell Widths of D2-to-D3 Co-Differentiated Myotubes and Comparison of Expression Levels of Key Proteins Involved in Skeletal Muscle Contraction and Relaxation on Day 5 after Initiation of Differentiation

The cell widths of D2-to-D3 co-differentiated myotubes and the expression levels of key proteins involved in skeletal muscle contraction and relaxation were compared on day 5 after initiation of differentiation.

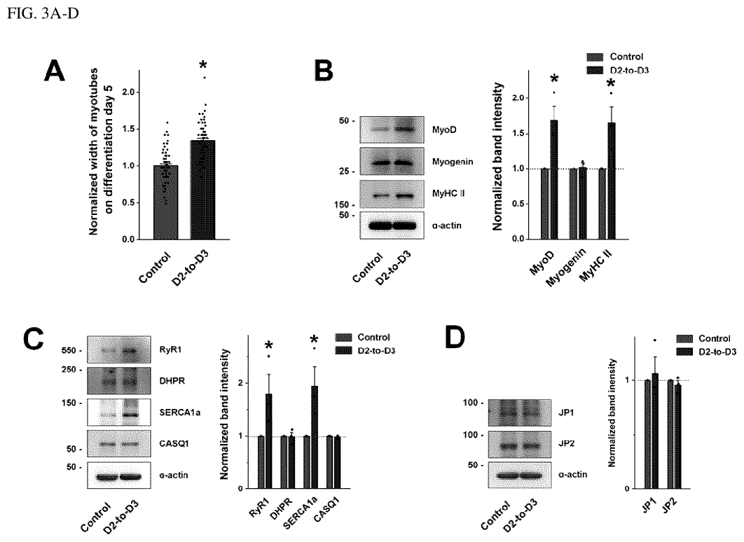

FIG. 3A and Table 5 show the results of measuring the width of D2-to-D3 co-differentiated myotubes on day 5 of differentiation, values for the control were set to 1, relative changes with respect to the values were expressed as normalized ratios, and the number of experiments used for analysis and statistics and the resulting values are shown in Table 5. *Significant differences compared to the control are indicated (P<0.05).

D2-to-D3 co-differentiated myotubes were thicker than control cells, indicating that terminal differentiation had been more successful, and this is supported by the quantitative increase of MyoD, a factor involved in terminal differentiation, and MyHC II, one of the skeletal muscle contractile proteins.

TABLE 5
Width of co-differentiated myotubes on day 5 of differentiation

| | Co-differentiation condition 1 | | | | Co-differentiation condition 2 | | | |
|---|---|---|---|---|---|---|---|---|
| | | Mb- | D2- | D3- | | Mb- | D2- | D3- |
| | Control | to-D2 | to-D2 | to-D2 | Control | to-D3 | to-D3 | to-D3 |
| Day 5 of | 1.00 ± | 0.87 ± | 0.93 ± | 0.97 ± | 1.00 ± | 0.86 ± | 1.35 ± | 1.14 ± |
| differentiation | 0.03 | 0.03 * | 0.03 | 0.04 | 0.03 | 0.03 * | 0.03 * | 0.03 * |

Next, immunoblot analysis was performed on day 5 to compare the expression levels of MyoD and myogenin (terminal differentiation-associated protein), MyHC II (contraction-mediating protein), and RyR1, DHPR, SERCA1a, and CASQ1, which are core proteins mediating skeletal muscle contraction and relaxation, and the expression levels of triad formation-mediating proteins in D2-to-D3 co-differentiated myotubes.

The expression level of α-actin, a sarcomeric structural protein of all cells, was confirmed (no change). CASQ1: calsequestrin 1, JP: junctophilin. Values for the control were set to 1, relative changes with respect to the values were expressed as normalized ratios, and the number of experiments used for analysis and statistics and the resulting values are shown in Table 6. The information on antibodies used is shown in Table 1. α-actin was used as a control to demonstrate that the total protein content of a sample loaded onto the gel remained unchanged. *Significant differences compared to the control are indicated (P<0.05).

As shown in FIGS. 3B to 3D and Table 6, the expression of RyR1 and SERCA1, which are key proteins in skeletal muscle contraction and relaxation, increased, consistent with the increased response to KCl, a substance that induces excitation-contraction coupling (Table 4). That is, it means that both the outward completion of terminal differentiation and the degree of physiological functional completion are higher in D2-to-D3 co-differentiated myotubes compared to the control.

TABLE 6
Comparison of the intensities of immunoblotting bands for differentiation
factor proteins, excitation-contraction-mediating proteins, and
triad formation-mediating proteins in lysates of co-differentiated
myotubes obtained on day 5 of differentiation

| | | Control | D2-to-D3 |
|---|---|---|---|
| Differentiation factors | MyoD | 1.00 ± 0.00 | 1.69 ± 0.20 * |
| and MyHC II | Myogenin | 1.00 ± 0.00 | 1.02 ± 0.07 |
| | MyHC II | 1.00 ± 0.00 | 1.65 ± 0.23 * |
| α-actin for differentiation | | 1.00 ± 0.00 | 1.00 ± 0.02 |
| factors and MyHC II | | | |
| Excitation-contraction- | RyR1 | 1.00 ± 0.00 | 1.80 ± 0.37 * |
| mediating proteins | DHPR | 1.00 ± 0.00 | 0.99 ± 0.08 |
| | SERCA1a | 1.00 ± 0.00 | 1.94 ± 0.37 * |
| | CASQ1 | 1.00 ± 0.00 | 0.98 ± 0.02 |
| α-actin for excitation- | | 1.00 ± 0.00 | 1.02 ± 0.04 |
| contraction-related proteins | | | |
| Triad formation- | JP1 | 1.00 ± 0.00 | 1.06 ± 0.15 |
| mediating proteins | JP2 | 1.00 ± 0.00 | 0.96 ± 0.04 |

| | | |
|---|---|---|
| α-actin for triad | 1.00 ± 0.00 | 1.0 ± 0.04 |
| formation-mediating proteins | | |

### <Experimental Example 4> Comparison of the Store-Operated Calcium Entry (SOCE) that Mediates External Calcium Procurement During Skeletal Muscle Contraction, the Cytosolic Calcium Concentrations, the Amounts of Calcium Stored in the Sarcoplasmic Reticulum, and the Expression Levels of SOCE-Mediating Proteins in D2-to-D3 Co-Differentiated Myotubes on Day 5 after Initiation of Differentiation

After the amount of calcium stored in the sarcoplasmic reticulum (SR) was depleted by thapsigargin (TG) treatment, the resulting store-operated calcium entry (SOCE) was measured, the amounts of calcium in the cytosol were compared, and the amounts of calcium stored in the sarcoplasmic reticulum were compared by thapsigargin (TG) treatment (or the amounts of calcium that can be released from the sarcoplasmic reticulum into the cytosol, that is, the amounts of calcium that can be used for skeletal muscle contraction). Values for the control were set to 1, relative changes with respect to the values were expressed as normalized ratios, and the number of experiments used for analysis and statistics and the resulting values are shown in parentheses in Table 7. *Significant differences compared to the control are indicated (P<0.05). The data obtained from the number of co-differentiated myotubes shown in parentheses was combined and expressed as ±SE. Values for the control were set to 1, relative changes with respect to the values were expressed as normalized ratios. *Significant differences compared to the control are indicated when P<0.05.

In addition, immunoblotting was performed to compare the expression levels of SOCE-mediating proteins, and the expression level of α-actin, a sarcomeric structural protein of all cells, was confirmed. Values for the control were set to 1, relative changes with respect to the values were expressed as normalized ratios, and the number of experiments used for analysis and statistics and the resulting values are shown in Table 8. Data obtained from three independent experiments was combined and expressed as ±SE, values for the control were set to 1, and relative changes with respect to the values were expressed as normalized ratios. α-actin was used as a control to demonstrate that the total protein content of a sample loaded onto the gel remained unchanged. *Significant differences compared to the control are indicated when P<0.05.

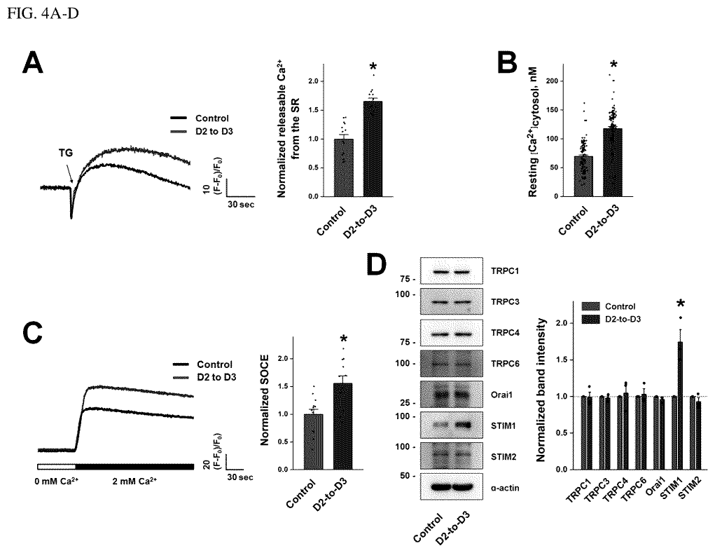

As shown in FIGS. 4A to 4D, the calcium influx from the outside into the cells in the D2-to-D3 co-differentiated myotubes was significantly increased compared to control cells, meaning that this is due to an increase in the SOCE-mediating protein STIM1. Furthermore, this means that the increased SOCE contributed to the increases in cytosolic calcium and the amount of calcium in the sarcoplasmic reticulum.

Overall, this means that the amount of calcium used for skeletal muscle contraction is increased and the resulting skeletal muscle contraction is more likely to occur.

TABLE 7
Measurement of store-operated calcium entry, cytosolic
calcium concentration, and amount of calcium stored
in the sarcoplasmic reticulum (SR) of co-differentiated
myotubes on day 5 of differentiation

| | Control | D2-to-D3 |
|---|---|---|
| Store-operated calcium | 1.00 ± 0.09 | 1.56 ± 0.14 * |
| entry (SOCE) | (85 myotubes | (86 myotubes |
| | from 14 wells) | from 14 wells) |
| Cytosolic | 69.38 ± 2.58 | 117.89 ± 3.71 * |
| [Ca²⁺], nM | (104 myotubes | (106 myotubes |
| | from 16 wells) | from 16 wells) |
| Amount of calcium that | 1.00 ± 0.08 | 1.65 ± 0.06 * |
| can be released from the | (94 myotubes | (98 myotubes |
| sarcoplasmic reticulum | from 14 wells) | from 14 wells) |
| (SR) | | |

TABLE 8
Comparison of the intensities of bands for SOCE-
mediating proteins by immunoblotting for lysates
of co-differentiated myotubes obtained on day 5.

| | Control | D2-to-D3 |
|---|---|---|
| TRPC1 | 1.00 ± 0.00 | 0.99 ± 0.08 |
| TRPC3 | 1.00 ± 0.00 | 0.98 ± 0.03 |
| TRPC4 | 1.00 ± 0.00 | 1.05 ± 0.12 |
| TRPC6 | 1.00 ± 0.00 | 1.03 ± 0.08 |
| Orai1 | 1.00 ± 0.00 | 0.96 ± 0.02 |
| STIM1 | 1.00 ± 0.00 | 1.74 ± 0.17 * |
| STIM2 | 1.00 ± 0.00 | 0.93 ± 0.05 |
| α-actin | 1.00 ± 0.00 | 1.0 ± 0.05 |

As described above, in transplanting myotubes into the sites of skeletal muscle atrophy/wasting caused by genetic or acquired skeletal muscle diseases, skeletal muscle atrophy/wasting that occurs during the long-term treatment of other diseases, or sarcopenia of skeletal muscle that occurs during the natural aging process, the present invention provides a solution to the problems of satellite stem cells, which have a quantitative disadvantage, or myoblasts, which have low success rates in proliferation and early terminal differentiation of cells transplanted at a transplanted site after transplantation.

When D2 immature myotubes, which have undergone partial terminal differentiation, are used as transferred cells, the cells transplanted at the transplanted site proliferate and are able to skip the early terminal differentiation process, so that by using D2 immature myotubes, which have been cultured and partially differentiated outside the human body (in vitro culture), as transferred cells, it is possible to overcome the low success rate of skeletal muscle cell transplantation, which is inevitably low due to the failure of proliferation and the early terminal differentiation process of transferred cells.

The use of D2 immature myotubes not only can increase the success rate of skeletal muscle cell transplantation, but also can increase skeletal muscle contractility (skeletal muscle physiological activity) by enhancing the ability to procure calcium for skeletal muscle contraction at the transplanted site when D3 immature myotubes are used as host cells and D2-to-D3 mature co-differentiation myotubes are used as transferred cells.

What is claimed is:

1. A method for preparing D2-to-D3 mature co-differentiation myotubes, the method comprising
co-differentiating D2 immature myotubes and D3 immature myotubes, wherein the D2 immature myotubes obtained on day 2 after initiation of terminal differentiation of myoblasts are used as transferred cells, and the D3 immature myotubes obtained on day 3 are used as host cells.

2. The method of claim 1, wherein the co-differentiation comprises transferring the D2 immature myotubes into a culture medium containing the D3 immature myotubes, and culturing them together.

3. The method of claim 1, wherein the myoblasts are selected from the group consisting of mouse myoblasts, rat myoblasts, frog myoblasts, rabbit myoblasts, guinea pig myoblasts, porcine myoblasts, monkey myoblasts, and human myoblasts.

4. The method of claim 1, wherein for the co-differentiation, D2 immature myotubes are transferred to D3 immature myotubes and then an additional differentiation culture is performed for two days.

5. The method of claim 1, further comprising selecting myotubes that have increased expression of MyoD, MyHC II, RyR1, SERCA1, and STIM1 proteins, increased cell thickness, and increased intracellular or extracellular calcium procurement for skeletal muscle contraction compared to control myotubes obtained by differentiating myoblasts in vitro for 5 days.
